(19) United States
(12) Patent Application Publication
(10) Pub. No.: US 2008/0221704 A1
(43) Pub. Date: Sep. 11, 2008

(54) Three (3) distinct improvements (different fields of use) to the prior art or invention entitled instant face-lifter

(76) Inventor: Ricardo Alfredo Fuenmayor Aray, San Carlos (VE)

Correspondence Address: Ricardo A. Fuenmayor A.;2nd Floor, 6318 23 Ave., Brooklyn, NY, 11204, US

(21) Appl. No.: 11/893,310

(22) Filed: Aug. 15, 2007

Related U.S. Application Data
(60) Provisional application No. 60/904,996, filed on Mar. 5, 2007.
(60) Provisional application No. 60/905,057, filed on Mar. 5, 2007.

(51) Int. Cl. A61F 2/10

(52) U.S. Cl. 623/23.72; 604/891.1

(57) ABSTRACT

There are three distinct improvements on the Instant Face-Lifter -the prior art-: Field of Use “A”, an intra-scalp round or oval-shaped device made of a biocompatible material, typically measuring 5 12/16″ long×4 12/16″ wide, stretches the face's skin, instantly producing a younger look, by injecting saline through a septum into an accordion-type side walls saline reservoir located inside the device thus allowing the user to control at will the amount of skin-stretching. Field of Use “B” uses an Instant Face-Lifter as a stand or support for an intra-scalp metering pump fitted with an in-vein catheter, a liquid drug reservoir, a septum, and a rechargeable battery, as to allow the automated daily intravenous supply of pharmaceutical drugs, thus circumventing the need of uncomfortable daily shots. Field of Use “C” uses an Instant Face-Lifter as a stand or support for an intra-scalp cellular phone or walkie-talkie fitted with rechargable batteries and sub-dermal headphone, microphone, and micro-switches, plus an external remote control/number pad to offer an all-weather, hands-free, instant, two-way communication between parties.

### TITLE OF THE IMPROVEMENTS OF INVENTION

I am submitting three (3) distinct improvements (different Fields of Use) to the prior art or invention entitled Instant Face-Lifter*:
Field of Use A: “Saline-inflatable Face-Lifter”: A Saline-Fillable Intra-scalp Face-Lifter with accordion-type flexible side walls.
Field of Use B: “Intra-scalp Drug Metering Pump Face-Lifter”: Using a Face-Lifter as a stand or support for a Refillable, Unnoticeable, Timer-driven, Remote-controlled, Intra-scalp, Pharmaceutical Drug Delivery Pump.
Field of Use C: “Intra-scalp Cell Phone Face-Lifter”: Using a Face-Lifter as a stand or support for an Implanted, Intra-scalp Cellular Phone and/or Walkie-Talkie.

(*Note: An “Instant Face-Lifter”, a beautifying device, consists of an intra-scalp oval-shaped device made of solid, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. ½″, ¾″, 1″, ½″, etc—at its center as to be able to provide the user the desired amount of skin stretching effect”)

### BACKGROUND OF THE IMPROVEMENTS OF THE INVENTION

The field of endeavor to which the invention pertains, based on the U.S. Patent Classification Definitions, is Class 128, Surgery, subclasses for apparatus which assists a body part or function but does not physically replace or partially replace any normally existing body part, and Sub-Class 899, devices placed entirely within body and means used therewith. This subclass is indented under subclass 897. Subject matter including devices not elsewhere classifiable which are placed entirely within the body either through insertion through natural body openings or by surgical implantation and means used therewith.

### BACKGROUND OF THE IMPROVEMENTS OF THE INVENTION: FIELD OF USE “A”

In the Field of Use “A”, the improvement on the prior art consists of a basic Face-Lifter to which it has been added: a) Expandable accordion-type side walls, b) an inflatable reservoir for saline solution, and c) a septum or injection port on top of the reservoir for letting in varying volumes of saline solution.

Rather than having a fixed height, this improvement allows the increase of the invention's final volume—therefore its height—, thus allowing the user to choose at will the desired amount of skin-stretching action. The injection port is a self-sealable round silicone septum (approx. 1″ diameter, 4 mm thickness) located on the upper side of the saline reservoir. The septum is easily located on the user's scalp, on the crown's area, by the sense of touch; it will feel as a circular soft spot. Deflated, the device's thickness measures half an inch. Once a variable volume of saline (between 150 and 300 ml) is injected into the fillable chambers, the device will increase its height up to an inch and a half, allowing for an extra skin-stretching action, thereby further reducing eyebrow wrinkles and providing an instant fresher look.

As the prior art, the improvement also consists of an oval-shaped device made of flexible medical grade silicone, typically measuring 5 12/16″ long×4 12/16″ wide, with sloping edges and an ever increasing thickness in the direction from border to center, starting at a negligible thickness at its borders and increasing to a maximum thickness of 1½″ at the center, once inflated with saline. The invention is also slided under the scalp, in the space between the skin of the scalp and the galea aponeurotica of the user through a 3″ to 4″ incision on the parietal area using local anesthesia.

### BACKGROUND OF THE IMPROVEMENTS OF THE INVENTION: FIELD OF USE “B”

In the Field of Use “B”, the improvement on the prior art consists of the use of a Face-Lifter as a stand or support for implanting a Refillable, Unnoticeable, Portable, Timer-driven, Remote-controlled, Intra-scalp Pharmaceutical Drug Delivery Pump. The improvement consists of a basic intra-scalp Face-Lifter, in whose silicone matrix has been embeded: a) a small flat metering pump, b) an inflatable reservoir with a septum or injection port (for storing a liquid drug), c) a rechargable battery (its energy provided by an electricity-producing motion-device similar to Seiko's® “Kinetic”® watches), and, c) an in-vein catheter. The flat metering pump can be controlled by means of a) an internal timer that can be set up to inject i.e. XX ml of a given drug every 8 hrs (diabetic patients, Human Growth Hormone for children, or drugs for ostheoporosis patients), or, b) activated at will by remote control (with a built-in clocked software so that it cannot be activated more than i.e. once every XX hours—to avoid overdosing, i.e. insuline—).

Advantages: the improvement provides a launching pad for a intra-scalp metering pump with an in-vein catheter, intended to help patients who require daily uptake of drug dosages: diabetics, osteoporotic people, children on Human Growth Hormone therapy, thus offering convenience (no more three shots/day, i.e. diabetes), instantaneous results for Viagra® users, and also to help control agressive patients (mental hospitals) or dangerous offenders (prison system) by supplying appropriate amounts of mind-control drugs, producing the desired effect in seconds. It may also raise self-steem by being hidden from view. The improvement is refillable through a self-sealable septum on the top of the head (1″ diameter, 4 mm thickness and located on the upper side of the liquid drug reservoir). For refilling, using a standard syringe, the septum is easily located on the user's scalp, on the crown's area, by the sense of touch; it will feel as a circular soft spot.

From internet:Total Prevalence of Diabetes in the US, All Ages, 2005

Total: 20.8 million people—7 percent of the population—have diabetes.

(Diagnosed: 14.6 million people Undiagnosed: 6.2 million people)

### BACKGROUND OF THE IMPROVEMENTS OF THE INVENTION FIELD OF USE “C”

In the Field of Use “C”, the improvement on the prior art consists of using the basic Face-Lifter as a support/stand for a portable communicating device, an Implanted, Intra-scalp, Cellular Phone or Walkie-Talkie.

Embedded into the silicone matrix of the Face-Lifter comes a) a flat cellular phone, b) a rechargable battery (its energy provided by an electricity-producing motion-device similar to Seiko's® “Kinetic”® watches), and c) a thin sub-dermal silicone side strap carrying wiring and electronic pieces on one of the sides of the face-lifter.

The sub-dermal silicone strap, 10″ long×½″ wide, implanted also under the skin, contains three separate sets of wiring and four distinct elements (See Drawing Sheet 3/3, FIG. 1, Section View). The sub-dermal strap carries: a) an embedded headphone and wiring right to the ear canal, and b) continuing its way down the temporal area, behind the jaw hinges, below the chin, until reaching the vocal cords area where it ends carrying a flat microphone and wiring; and, c) two (2) flat, membrane-type, micro-switches: One ON-OFF silencer switch (for privacy) and another switch (“Send”) for passing the calls without the need of using the remote control.

A remote control/number pad is supplied along the device (dialing numbers, Send button, etc.) which allows to dial numbers, make phone calls, etc.

Advantages: Using the basic Face-Lifter as a support or stand for an intra-scalp, implanted Cellular phone or Walkie-Talkie would be useful for: Team sports (manager needs to talk to the players throughout a game), people in Finance (stockbrokers who need up-to-the-minute share information), politicians, any cell phone users (hands-free communication under all-type-of-weather, goes undetected, two-way instant communication), police, firemen, etc., while simultaneously smoothening out wrinkles and producing a fresher and younger look to the user.

### BRIEF SUMMARY OF THE IMPROVEMENT ON INVENTION

Field of Use A, the improvement consists of a basic Face-Lifter to which it has been added: a) expandable accordion-type side walls, b) an inflatable reservoir for saline solution, and c) an injection port on top of the reservoir for letting in varying volumes of saline. This improvement allows the user to increase the Face-Lifter height at will by selecting the volume of saline injected in the reservoir, thus allowing the user to choose at will the desired amount of skin-stretching action of the device.

Field of Use B, the improvement consists of the use of a basic Intra-scalp Face-Lifter as a stand/support for a pharmaceutical drug metering pump plus a refillable reservoir with septum, a rechargable battery, and an in-vein catheter. The pump can be controlled by a timer or activated at will by remote control.

Field of Use C, the improvement consists of the use of a basic Intra-scalp Face-Lifter as a stand/support for a Cellular Phone and/or Walkie-Talkie plus a rechargable battery and a sub-dermal side strap which carries a flat headphone, a microphone, and two micro-switches and wiring, allowing the user to carry on a two-way communication with other party in a hands-free fashion and under all kind of weather (even while swimming in a pool!).

### BRIEF DESCRIPTION OF THE SEVERAL VIEWS OF THE DRAWINGS

Listing of figures: One Sheet of Drawings for each Field of Use: A, B and C.

Field of Use A: Sheet 1/3. 2 Figures.

FIG. 1. Section View

It corresponds to a sectional cut through the middle of the device along its longest axis. It consists of a saline fillable chamber, a septum/injection port, and accordion-type side walls, all the structures being embedded in the silicone (or other biocompatible inert material) matrix of the Face-Lifter, its purpose being to stretch the skin of the user's face at will by adjusting the volume of saline injected into the device, thus smoothening out wrinkles and producing a softer, younger look.

FIG. 2. Elevation View

This drawing corresponds to the depiction of the invention as seen from above. The curved lines represent an ever increasing thickness starting from negligible thickness at the borders to a maximum thickness of 1½″ at the center of the device, when totally filled with saline.

Field of Use B: Sheet 2/3. 2 Figures.

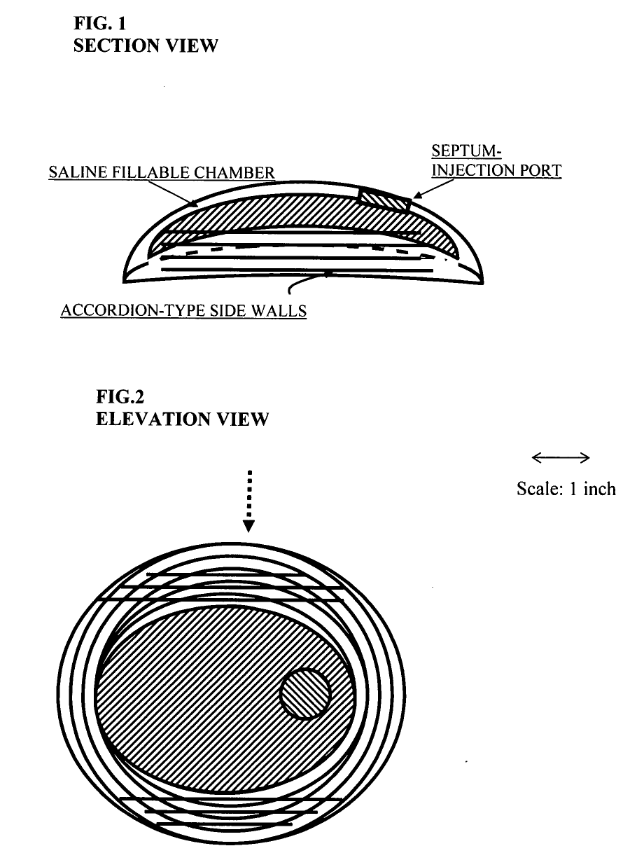

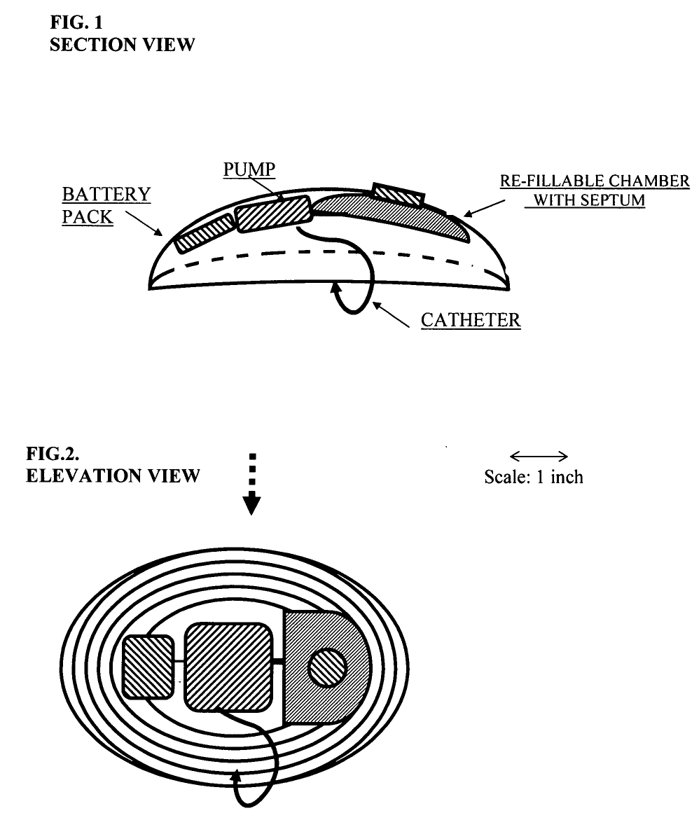

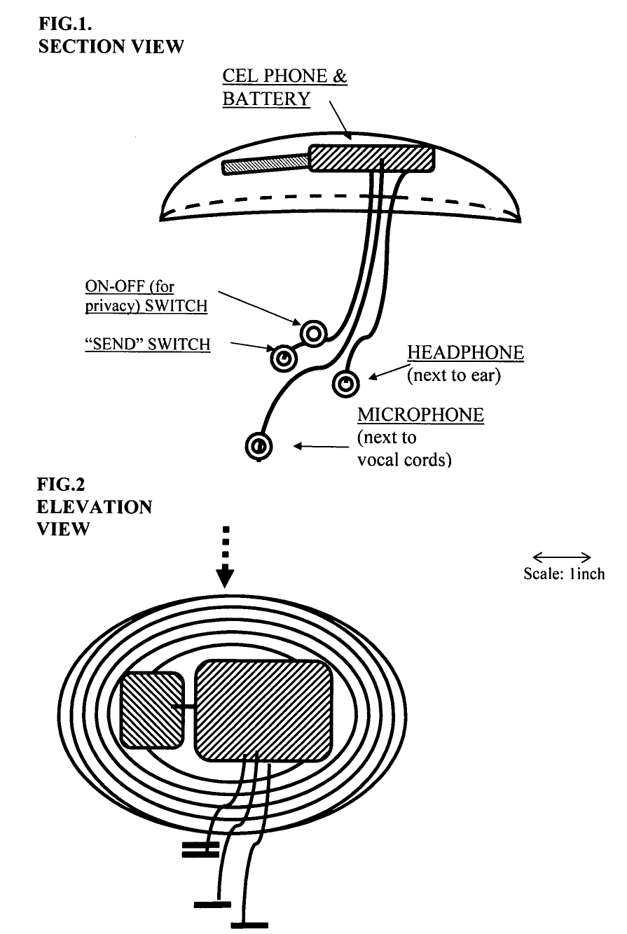

FIG. 1. Section View

It corresponds to a sectional cut through the middle of the device along its longest axis. The improvement consists of the use of a basic Face-Lifter as a stand/support for a flat metering pump provided with a refillable chamber with septum, a rechargable battery, and an in-vein catheter, all the structures embedded in the silicone (or other biocompatible inert material) matrix of the device, its purpose being to intravenously supply the required amount of pharmaceutical drug via the catheter while eliminating the need of continuous shots, additionally getting a younger, fresher look.

FIG. 2. Elevation View

This drawing corresponds to the depiction of the invention as seen from above showing a Face-Lifter used as a stand/support for a flat metering pump, a refillable chamber with septum, a rechargable battery and an in-vein catheter.

Field of Use C: Sheet 3/3. 2 Figures.

FIG. 1. Section View

It corresponds to a sectional cut through the middle of the device along its longest axis. It consists of an improvement over prior art consisting of the use of a Face-Lifter as a stand/support for an intra-scalp flat cellular phone, a rechargable battery and three wirings with electronics embedded in a silicone side strap. The sub-dermal side strap carries an embedded headphone and wiring next to the ear, a flat microphone and wiring next to the vocal cords area, and two flat, membrane-type, micro-switches on the temporal area: One ON-OFF silencer switch (for privacy) and another switch (“Send”) for passing the calls without the need of using the remote control

FIG. 2. Elevation View

This drawing corresponds to the depiction of the proposed improvement of invention as seen from above. The improvement consists of the use of a Face-Lifter as a stand/support for a flat cellular phone and/or a Walkie-Talkie with a rechargable battery and a sub-dermal strap carrying a headphone, a microphone, two flat membrane-type switches and wiring.

### DETAILED DESCRIPTION OF THE IMPROVEMENTS OF AN INVENTION

I am submitting three (3) distinct improvements (covering different Fields of Use) to the prior art or invention entitled Instant Face-Lifter:
Field of Use A: “Saline-inflatable Face-Lifter”: A Saline-Fillable Intra-scalp Face-Lifter with accordion-type flexible side walls.
Field of Use B: “Intra-scalp Drug Metering Pump Face-Lifter”: Using a Face-Lifter as a stand or support for a Refillable, Unnoticeable, Timer-driven, Remote-controlled, Intra-scalp, Pharmaceutical Drug Delivery Pump.
Field of Use C: “Intra-scalp Cell Phone Face-Lifter”: Using a Face-Lifter as a stand or support for an Implanted, Intra-scalp Cellular Phone and/or Walkie-Talkie.

In all three cases or improvements, they share as common ground the basic structure and design of the Instant Face-Lifter*, keeping in all three cases its main feature of being a beautifying device that stretches the skin of the user's face producing a younger look, while adding to its usefulness by serving as a stand or support for other pieces of equipment, In all three improvements, the invention is slided under the scalp of the user through a 3″ to 4″ long horizontal incision on the parietal area using local anesthesia.

### DETAILED DESCRIPTION OF THE IMPROVEMENT: FIELD OF USE “A”

In the Field of Use A, the improvement on the prior art consists of a basic Face-Lifter (“which consists of an intra-scalp oval-shaped device made of solid, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. ½″, ¾″, 1″, 1½″, etc—at its center as to be able to provide the user the desired amount of skin stretching effect”) to which it has been added: 1) expandable (accordion-type) side walls, 2) an inflatable reservoir for saline solution, and 3) a septum as injection port, the latter located on top of the saline reservoir.

Instead of having a fixed height as the prior art, this improvement, by means of injecting varying volumes of saline, allows the user to control at will the invention's final height, as to allow him/her to choose the desired amount of skin-stretching action.

The improvement includes a saline injection port: a self-sealable round silicone septum (1″ diameter, 4 mm thickness) located on the upper side of the saline reservoir. The user can easily identify the septum's location by the sense of touch, feeling it as a circular soft spot. Once the desired volume of saline is injected into the fillable chambers, the device will increase its height up to an inch and a half, allowing for an extra skin-stretching action, thereby further reducing frown lines and lifting the eyebrows thus producing an instant and permanent fresher and younger look.

### DETAILED DESCRIPTION OF THE IMPROVEMENTS: FIELD OF USE “B”

In the Field of Use B, the improvement on the prior art consists of the use of a Face-Lifter as a stand or support for a refillable, intra-scalp, pharmaceutical drug delivery pump and an in-vein catheter. The improvement consists of the basic intra-scalp Face-Lifter, which has, embeded in its silicone matrix, a small flat metering pump, an inflatable liquid drug reservoir with a septum, a rechargable battery, and an in-vein catheter. The metering pump can be controlled internally by means of a timer (that can be set up to inject i.e. XX ml of a given drug every 8 hrs, in the case of diabetic patients, children under Human Growth Hormone therapy, or drugs for ostheoporosis patients), or, it can be externally activated by remote control (that carries a built-in clocked software so that it cannot be activated more than i.e. once every XX hours—to avoid overdosing i.e. insuline—).

There is currently a large number of people who suffer from diabetes in the US (20.8 million people—7 percent of the population—have diabetes). To keep alive, many of them have to endure a daily ordeal of multiple intramuscular insuline shots. Women with ostheoporosis must take daily doses of pills that irritate the gastrointestinal mucus, producing lots of pain. One of the improvement's goals is to eliminate the painful need of multiple daily shots and to circumvent the pain associated with daily ingestions of drugs that produce gastrointestinal irritation. Furthermore, a drug injected intravenously takes less than 20 seconds to be distributed all over the body: this is advantageous for Viagra® users who can feel the effect of the drug immediately by simply activating a button of a remote control. Thus, the improvement may also raise self-steem by being hidden from view. This improvement may help control agressive patients (mental hospitals) or dangerous offenders (prison system) by supplying appropriate amounts of mind-control drug, producing the desired effect in seconds.

The improvement is refillable through a self-sealable septum located on top of the head (1″ diameter, 4 mm thickness and located on the upper side of the liquid drug reservoir). A standard syringe is used for refilling. The septum is easily located on the user's scalp, on the crown's area, by the sense of touch; it will feel as a circular soft spot.

### DETAILED DESCRIPTION OF THE IMPROVEMENTS: FIELD OF USE “C”

In the Field of Use C, the improvement consists in the use of a Face-Lifter as a stand/support for a portable communicating device: an Implanted, Intra-scalp. Cellular Phone.

Embedded into the silicone matrix of the Face-Lifter comes a) a flat cellular phone, b) a rechargable battery (its energy provided by an electricity-producing motion-device similar to Seiko's® “Kinetic”® watches), and c) a thin sub-dermal silicone side strap with electronics on one of the sides of the Face-Lifter.

The silicone strap, measuring 10″ long×½″ wide, implanted under the skin, contains three separate sets of wiring and four distinct elements (See Drawing Sheet 3/3, FIG. 1, Section View). The sub-dermal strap carries: a) an embedded headphone and wiring right to the ear canal, and b) continuing its way down the temporal area, behind the jaw hinges, below the chin, until reaching the vocal cords area where it ends carrying a flat microphone and wiring; and, c) two (2) flat, membrane-type, micro-switches: One ON-OFF silencer switch (for privacy) and another switch (“Send”) for passing the calls without the need of using the remote control.

A remote control is supplied accompanying the device (dialing numbers, Send button, etc.) which allows to dial numbers, make phone calls, etc.

Among its advantages, the intra-scalp, implanted Cell phone would be useful for: Team sports (manager needs to talk to the players throughout a game), people in Finance (stockbrokers who need up-to-the-minute share information), politicians, any cell phone users (hands-free communication under all-type-of-weather, goes undetected, two-way instant communication), police, firemen, etc., while simultaneously smoothening out wrinkles and producing a fresher and younger look to the user.

What is claimed is:

1. Field of Use “A”: An intra-scalp round or oval-shaped device made of a biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of adding to it accordion-type flexible side walls, an inflatable saline solution reservoir and a saline injection port located on top of the reservoir wherein, thus allowing the user to control the device's final height by regulating the volume of saline injected into the inflatable saline solution reservoir therefore letting the user choose the desired amount of skin-stretching action for rejuvenation purposes.

2. The device according to claim 1, wherein the saline injection port is a self-sealable round silicone septum located on the upper side of the saline solution reservoir.

3. The device according to claim 1, wherein the biocompatible material is medical-grade silicon or other inert synthetic or naturally derived polymeric material or substance.

4. Field of Use “B”: An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of embedding into the device's structure means for allowing the automated and timed intravenous supply of pharmaceutical drugs said means comprising:
a small flat metering pump
an in-vein catheter
a liquid drug reservoir with a septum and
a rechargeable battery.

5. The device according to claim 4, wherein the metering pump can be controlled internally by means of a timer.

6. The device according to claim 4, wherein the metering pump can be externally activated by remote control thus allowing patients who must be administered daily drug dosages (i.e. diabetic people, children under Human Growth Hormone therapy, ostheoporotic patients, and mental patients under mind-controlling drugs) to circumvent the need of uncomfortable daily shots or avoiding complications related to the continuous ingestion of mucus irritating-pills.

7. Field of Use “C”: An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of embedding into the device's structure a portable communicating device.

8. The device according to claim 7, wherein the communicating device is an implanted cellular phone.

9. The device according to claim 7, wherein the communicating device is an implanted walkie-talkie.

10. The device according to claim 7-8-9, further comprising:
a rechargable battery and
a thin sub-dermal silicone side strap fitted with
an embedded headphone and wiring leading to the ear canal,
a flat microphone and wiring leading to the vocal cords area,
a membrane-type micro-switch for ON-OFF function
a membrane-type micro-switch for passing the calls and
an external remote control/number pad thus providing an all-weather, hands-free, instant, two-way communication between parties.
